(19) United States
(12) Patent Application Publication
(10) Pub. No.: US 2009/0111117 A1
(43) Pub. Date: Apr. 30, 2009

(54) DEVICE AND METHOD FOR DETECTING COMPLEX FORMATION

(75) Inventors: Jae-Hyun Chung, Bellevue, WA (US); Kyong-Hoon Lee, Ann Arbor, MI (US); Kieseok Oh, Seattle, WA (US); Island Pinnick, Bend, OR (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: UNIVERSITY OF WASHINGTON, Seattle, WA (US)

(21) Appl. No.: 12/259,154

(22) Filed: Oct. 27, 2008

Related U.S. Application Data
(60) Provisional application No. 60/983,079, filed on Oct. 26, 2007.

(51) Int. Cl. C12Q 1/68; G01N 33/53; G01N 33/573; G01N 33/567

(52) U.S. Cl. 435/6; 435/7.1; 435/7.4; 435/7.2

(57) ABSTRACT

The present invention provides devices and methods for measuring electrically detectable bulk properties of liquid samples. Representative electrically detectable bulk properties measurable by the devices and methods of the invention include resistivity (conductivity) and dielectric constant (permittivity). The electrically detectable bulk properties are determined by comparing the experimental electrical output of the devices with mathematically simulated models of the experimental devices.

### CROSS-REFERENCE TO RELATED APPLICATION

This application claims the benefit of U.S. Provisional Patent Application No. 60/983,079, filed Oct. 26, 2007, expressly incorporated herein by reference in its entirety.

### BACKGROUND

Diseases worldwide spread rapidly as the use of transportation continues to increase. A simple, rapid, and reliable immunoassay format is required to diagnose and detect diseases in order to treat them as quickly as possible.

The majority of current hand-held kits are either latex agglutination tests or immunochromatographic lateral flow assays, which offer a reasonable sensitivity and specificity. These immunoassays, however, require immobilization of antibodies onto a solid support and also require the labeling of reagents with markers such as organic dyes and colloidal metal micro/nano-particles. As a result, such immunoassays are relatively expensive and have a limited shelf life even under refrigeration.

Label-free immunoassays have been attempted using both electromagnetic and electrochemical methods. However, both types of label-free assays suffer from requiring large and expensive equipment to perform and, thus, do not provide a cost-efficient, portable, and sensitive label-free immunoassay.

In order to satisfy the rapid and inexpensive detection of diseases throughout the world, a fast, inexpensive, and efficient label-free immunoassay is desired.

### SUMMARY OF THE INVENTION

The present invention provides devices and methods for measuring electrically detectable bulk properties of liquid samples. Representative electrically detectable bulk properties measurable by the devices and methods of the invention include resistivity (conductivity) and dielectric constant (permittivity).

In one aspect, the invention provides devices and methods for measuring resistivity (conductivity) or dielectric constant (permittivity) of liquid samples.

In one embodiment, the method for measuring the resistivity of a sample solution comprises providing a testing apparatus defining a fluidic structure on a substrate, the fluidic structure having a surface with a first electrode, a second electrode, and an electrode gap therebetween; introducing a fluid to the fluidic structure; applying a voltage across the first and second electrodes; measuring a transient electrical response to the applied voltage; estimating a resistance of the fluid based on the transient electrical response to the applied voltage; and calculating a resistivity of the fluid based on the estimated fluid resistance.

In another aspect, the invention provides devices and methods for detecting a complex formed by the binding interaction between first and second binding partners.

In one embodiment, the method for detecting a complex comprises providing a testing apparatus defining a well on a substrate, the fluidic structure having a surface with a first electrode, a second electrode, and an electrode gap therebetween; introducing a sample solution into the well; applying a voltage across the first and second electrodes; measuring a transient electrical response to the applied voltage; estimating a fluid resistance between the electrodes based on the measured transient electrical response; calculating a resistivity of the sample solution based on the estimated fluid resistance; and using the calculated resistivity of the sample solution to determine if a complex has been formed in the sample solution.

In one embodiment, the method for detecting a complex includes performing a label-free immunoassay comprising by the steps of: providing a testing apparatus defining a well on a substrate, the fluidic structure having a surface with a first electrode, a second electrode, and an electrode gap therebetween; introducing a sample solution into the well; applying a voltage across the first and second electrodes; measuring a transient electrical response to the applied voltage; estimating a fluid resistance between the electrodes based on the measured transient electrical response; calculating a resistivity of the sample solution based on the estimated fluid resistance; and using the calculated resistivity of the sample solution to determine if antigens are present in the sample solution.

In another embodiment, the method for detecting a complex comprises combining a first binding partner with a sample to provide a mixture, wherein the mixture comprises a complex formed by a binding interaction between the first binding partner and a second binding partner when the sample comprises the second binding partner; measuring an electrically detectable bulk property of the mixture; and determining the presence or absence of the complex in the mixture, and thereby the presence or absence of the second binding partner in the sample, based on the electrically detectable bulk property. Determining the presence or absence of the complex in the mixture based on the electrically detectable bulk property can include comparing the measured electrically detectable bulk property to an electrically detectable bulk property of one or more samples having known compositions. In one embodiment, the method for detecting the formation of a complex is an immunoassay. In this embodiment, the first binding partner is an antibody or fragment thereof and the second binding partner is an antigen, or the first binding partner is an antigen and the second binding partner is an antibody or fragment thereof. In another embodiment, the method for detecting the formation of a complex is a nucleic acid hybridization assay. In this embodiment, the first binding partner is a first nucleic acid and the second binding partner is a second nucleic acid.

In another aspect, the invention provides devices and methods for determining particle count in a sample that includes particles. In one embodiment, the method for determining a particle count comprises measuring an electrically detectable bulk property of a mixture comprising a plurality of particles; and determining a particle count based on the electrically detectable bulk property. Determining the particle count based on the electrically detectable bulk property can include comparing the measured electrically detectable bulk property to an electrically detectable bulk property of one or more samples having known particle counts.

In another aspect, the invention provides a method for determining the concentration of an analyte, comprising measuring an electrically detectable bulk property of a sample comprising an analyte; and determining the analyte concentration based on the electrically detectable bulk property. Determining the analyte concentration based on the electrically detectable bulk property can include comparing the measured electrically detectable bulk property to an electrically detectable bulk property of one or more samples having known analyte concentrations.

In a further aspect, the invention provides a method for screening therapeutic drug candidates for their ability to bind to a therapeutic target of interest. In one embodiment, the method includes combining a therapeutic drug candidate with a therapeutic target of interest to provide a mixture, wherein the mixture comprises a complex formed between the drug candidate and the target when the drug candidate has a binding interaction with the target; measuring an electrically detectable bulk property of the mixture; and determining the presence or absence of the complex in the mixture, and thereby the binding interaction between the drug candidate and the target, based on the electrically detectable bulk property. Determining the presence or absence of the complex in the mixture based on the electrically detectable bulk property can include comparing the measured electrically detectable bulk property to an electrically detectable bulk property of one or more samples having known compositions.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a partially exploded, fragmentary view of a representative device in accordance with the present invention;

FIG. 1B is a perspective view of the device shown in FIG. 1;

FIG. 2A is a diagrammatic cross-sectional view of a portion of the device shown in FIG. 1, shown in use;

FIG. 2B is a circuit diagram use to model the device shown in FIG. 1;

FIG. 3 illustrates graphically a least squares fit of experimentally obtained transient voltage data for pure water and the fitted curve generated by the model of the circuit diagram shown in FIG. 2B;

FIG. 4A is a micrograph of a representative device in accordance with the invention and the agglutination of antibody-coated polystyrene beads by immunoreactions between antibody-antigens;

FIG. 4B is a graph illustrating the root-mean-square voltage measured using a device in accordance with the present invention for various concentrations of an antigen;

FIGS. 5A-5C are micrographs illustrating devices in accordance with the present invention having electrodes positioned in various locations within a well;

FIG. 6 is a graph showing capacitance and resistance measurements taken from the devices illustrated in FIGS. 5A-5C;

FIG. 7 illustrates the change in the RMS values for antibody-antigen samples measured using the devices illustrated in FIGS. 5A-5C;

FIG. 8A a portion of a representative device in accordance with the present invention having a microchannel passing over a pair of electrodes;

FIGS. 8B-8C are micrographs showing the device of FIG. 8A having various concentrations of microspheres suspended in fluid passing through the channel and above the electrodes;

FIGS. 9A-9B are graphs showing the testing of various samples using a device in accordance with the present invention;

FIG. 10 illustrates a microfluidic system incorporating a device in accordance with the present invention;

FIG. 11 illustrates a multiplexed microfluidic system incorporating a device in accordance with the present invention; and

FIG. 12 illustrates a probe-based system incorporating a device in accordance with the present invention.

### DETAILED DESCRIPTION OF THE INVENTION

The present invention provides devices and methods for measuring electrically detectable bulk properties of liquid samples. Representative electrically detectable bulk properties measurable by the devices and methods of the invention include resistivity (and the related property of conductivity) and dielectric constant (and the related property of permittivity).

In one aspect, the invention provides devices and methods for measuring resistivity (conductivity) or dielectric constant (permittivity) of liquid samples. In one embodiment, the method for measuring the resistivity of a sample solution comprises providing a testing apparatus defining a fluidic structure on a substrate, the fluidic structure having a surface with a first electrode, a second electrode, and an electrode gap therebetween; introducing a fluid to the fluidic structure; applying a voltage across the first and second electrodes; measuring a transient electrical response to the applied voltage; estimating a resistance of the fluid based on the transient electrical response to the applied voltage; and calculating a resistivity of the fluid based on the estimated fluid resistance.

In one embodiment of the method, estimating the fluid resistance between the electrodes comprises comparing the transient electrical response to transient electrical responses calculated from an analytical model of the testing apparatus.

In another aspect, the invention provides devices and methods for detecting a complex formed by the binding interaction between first and second binding partners. In one embodiment, the method for detecting a complex comprises providing a testing apparatus defining a well on a substrate, the fluidic structure having a surface with a first electrode, a second electrode, and an electrode gap therebetween; introducing a sample solution into the well; applying a voltage across the first and second electrodes; measuring a transient electrical response to the applied voltage; estimating a fluid resistance between the electrodes based on the measured transient electrical response; calculating a resistivity of the sample solution based on the estimated fluid resistance; and using the calculated resistivity of the sample solution to determine if a complex has been formed in the sample solution.

In one embodiment of the method, estimating the fluid resistance between the electrodes comprises comparing the transient electrical response to transient electrical responses calculated from an analytical model of the testing apparatus.

In one embodiment of the method, the analytical model of the testing apparatus includes a capacitance modeling the electrical double layer capacitance at the electrodes.

In one embodiment of the method, the analytical model of the testing apparatus further includes a capacitance modeling the parasitic capacitance.

In one embodiment of the method, using the calculated resistivity of the sample solution to determine if a complex has been formed in the sample solution comprises comparing the calculated fluid resistivity to a predetermined fluid resistivity correlation.

In one embodiment of the method, the predetermined fluid resistivity correlation comprises a correlation of the resistivity of a control solution that is determined by measuring a transient electrical response of the control solution to an applied voltage and estimating a resistivity of the control solution based on the transient electrical response to the applied voltage.

In one embodiment of the method, the measured transient electrical response is the transient voltage.

In a further embodiment of the method, the step of selecting a parameter of the transient voltage selected from one of the transient voltage RMS value, peak voltage, transition voltage and steady state voltage, and using the selected parameter to determine if a complex has been formed in the sample solution.

In one embodiment, the method for detecting a complex includes performing a label-free immunoassay comprising by the steps of: providing a testing apparatus defining a well on a substrate, the fluidic structure having a surface with a first electrode, a second electrode, and an electrode gap therebetween; introducing a sample solution into the well; applying a voltage across the first and second electrodes; measuring a transient electrical response to the applied voltage; estimating a fluid resistance between the electrodes based on the measured transient electrical response; calculating a resistivity of the sample solution based on the estimated fluid resistance; and using the calculated resistivity of the sample solution to determine if antigens are present in the sample solution.

In another embodiment, the method for detecting a complex comprises combining a first binding partner with a sample to provide a mixture, wherein the mixture comprises a complex formed by a binding interaction between the first binding partner and a second binding partner when the sample comprises the second binding partner; measuring an electrically detectable bulk property of the mixture; and determining the presence or absence of the complex in the mixture, and thereby the presence or absence of the second binding partner in the sample, based on the electrically detectable bulk property. Determining the presence or absence of the complex in the mixture based on the electrically detectable bulk property can include comparing the measured electrically detectable bulk property to an electrically detectable bulk property of one or more samples having known compositions. In one embodiment, the one or more samples having known compositions have a known amount of the first binding agent. In one embodiment, the electrically detectable bulk property is conductivity or resistivity. In another embodiment, the electrically detectable bulk property is dielectric constant or permittivity.

In the methods, the property is measured by a cell that includes a pair of electrodes (first and second electrodes) separated by an electrode gap. The sample to be analyzed is introduced into the cell in electrical communication with the first and second electrodes. The property of the sample is measured by applying a voltage across the electrodes. The measurement is used to determine, for example, the formation or absence of the complex based on the electrically detectable bulk properties of the sample. Representative electrically detectable bulk properties measured in the method and by the device of the invention include conductivity, resistivity, dielectric constant and permittivity. Details of the method and device useful in carrying out the method are described below.

In one embodiment, the first binding partner is an antibody or fragment thereof and the second binding partner is an antigen. In another embodiment, the first binding partner is an antigen and the second binding partner is an antibody or fragment thereof. The antigen can be a small molecule, peptide, protein, polynucleotide, or polysaccharide.

In one embodiment, the first binding partner is a first nucleic acid and the second binding partner is a second nucleic acid. The first and second nucleic acids are independently selected from the group consisting of DNAs and RNAs.

In one embodiment, the first binding partner is an enzyme and the second binding partner is a substrate. In another embodiment, the first binding partner is a substrate and the second binding partner is an enzyme.

In one embodiment, the first binding partner is a receptor and the second binding partner is a ligand for the receptor. In another embodiment, the second binding partner is a receptor and the first binding partner is a ligand for the receptor.

In one embodiment, the first binding partner is a nucleic acid and the second binding partner is a protein. In another embodiment, the first binding partner is a protein and the second binding partner is a nucleic acid.

In one embodiment, the first binding partner is a cell, cell membrane, or organelle, and the second binding partner is a ligand for the cell, cell membrane, or organelle. In another embodiment, the second binding partner is a cell, cell membrane, or organelle, and the first binding partner is a ligand for the cell, cell membrane, or organelle.

In one embodiment, the first binding partner is immobilized on a solid phase.

In another aspect, the invention provides devices and methods for determining particle count in a sample that includes particles. In one embodiment, the method for determining a particle count comprises measuring an electrically detectable bulk property of a mixture comprising a plurality of particles; and determining a particle count based on the electrically detectable bulk property. Determining the particle count based on the electrically detectable bulk property can include comparing the measured electrically detectable bulk property to an electrically detectable bulk property of one or more samples having known particle counts. In one embodiment, the electrically detectable bulk property is conductivity or resistivity. In another embodiment, the electrically detectable bulk property is dielectric constant or permittivity. Representative particles that can be counted by the method of the devices and methods of the invention include nanoparticles (e.g., metal nanoparticles such as gold nanoparticles), microparticles (e.g., microspheres including polymeric microspheres), nucleic acids and nucleic acid particles, proteins and protein particles, viruses and virus particles, and cells including bacteria and cultured cells. The devices and methods of the invention can provide particle counts for samples containing from about 100 particles/mL to greater than about 1,000,000 particles/mL.

In another aspect, the invention provides a method for determining the concentration of an analyte, comprising measuring an electrically detectable bulk property of a sample comprising an analyte; and determining the analyte concentration based on the electrically detectable bulk property. Determining the analyte concentration based on the electrically detectable bulk property can include comparing the measured electrically detectable bulk property to an electrically detectable bulk property of one or more samples having known analyte concentrations. In one embodiment, the electrically detectable bulk property is conductivity or resistivity. In another embodiment, the electrically detectable bulk property is dielectric constant or permittivity.

In a further aspect, the invention provides a method for screening therapeutic drug candidates for their ability to bind to a therapeutic target of interest. In one embodiment, the method includes combining a therapeutic drug candidate with a therapeutic target of interest to provide a mixture, wherein the mixture comprises a complex formed between the drug candidate and the target when the drug candidate has a binding interaction with the target; measuring an electrically detectable bulk property of the mixture; and determining the presence or absence of the complex in the mixture, and thereby the binding interaction between the drug candidate and the target, based on the electrically detectable bulk property. Determining the presence or absence of the complex in the mixture based on the electrically detectable bulk property can include comparing the measured electrically detectable bulk property to an electrically detectable bulk property of one or more samples having known compositions. In one embodiment, the electrically detectable bulk property is conductivity or resistivity. In another embodiment, the electrically detectable bulk property is dielectric constant or permittivity.

In one embodiment, the target of interest is a protein and the therapeutic drug candidate is a small molecule, a peptide, a nucleic acid, or a polysaccharide.

In one embodiment, the target of interest is an enzyme and the therapeutic drug candidate is a substrate.

In one embodiment, the target of interest is a receptor and the therapeutic drug candidate is a ligand for the receptor.

In one embodiment, the target of interest is nucleic acid and the therapeutic drug candidate is a protein or peptide.

In one embodiment, the target of interest is a cell, cell membrane, or organelle and the therapeutic drug candidate is a ligand for the cell, cell membrane, or organelle.

In one embodiment, the target of interest is immobilized on a solid phase.

For the methods and devices of the invention that involve detecting complex formation between a first and second binding partner, it will be appreciated that there are several ways to combine the first (e.g., antibody or therapeutic target of interest) and second (e.g., antigen or therapeutic drug candidate) binding partners. The first binding partner can be present in the device (e.g., well) prior to the addition of a sample (containing, for example, antigen or therapeutic drug candidate) to be analyzed. For example, the well can include a lyophilized antibody or therapeutic target, optionally immobilized on a solid phase, such as a bead or particle (nano- or microparticle). Alternatively, the first binding partner (optionally immobilized on a solid phase) can be added to the sample prior to its introduction into the device well. In a further alternative, the first binding partner can be added to the sample after the sample has been introduced into the well.

The devices and methods of the invention can be used to provide kinetic information. Because the devices and methods of the invention provide for measuring an electrically detectable property of the sample by applying a voltage across first and second electrodes and then measuring the transient response to the applied voltage, the devices and methods can interrogate a sample periodically as a function of time and provide kinetic information. By virtue of the nature of the devices and methods of the invention, the devices and methods can provide kinetic information regard, such as the rate of complex formation between first and second binding partners.

The advantages of the devices and methods of the invention relating to providing kinetic information are not limited to monitoring complex formation between first and second binding partners. The devices and methods can provide kinetic information for samples (e.g., reaction solutions or mixtures) in which chemical, biological, or biochemical reactions take place and provide information regarding these reactions, such as reaction rate, reaction completion, or the presence of absence of desired or undesired reaction products. The devices and methods of the invention can be advantageously used in process monitoring including, for example, the chemical/biochemical reactions noted above, as well as monitoring the formation of products in fermentation processes (e.g., recombinant proteins) and cell production in cell culturing processes.

The following definitions are useful in understanding the invention.

As used herein, “bulk property” means a measurable property of a mixture (sample or control) that is affected by more than one and up to all of the individual components of the mixture being measured (i.e., a property to which more than one and up to all of the individual components contribute a measurable signal rather than being determined by only one of the components without significant, measurement-affecting contributions from other components that are present). The measurement of individual components of a potential binding interaction, either after separation or by use of a signal that is selective for a particular component, is not a bulk property measurement because only one component at a time is responsible for the property being measured. For example, measurement of absorbance at a wavelength that is absorbed by only one of the three components—ligand, antiligand, and complex—present in a binary equilibrium mixture, the wavelength being selected so that contributions to the signal of other components in the mixture can be ignored, is not considered to be a bulk property measurement.

The present invention relates to “electrically detectable” bulk properties, bulk properties that can be measured using an electrical signal that interacts with the sample, followed by detection of the signal as modified by the sample. Examples of electrically detectable bulk properties include resistivity, conductivity, dielectric constant, and permittivity. The properties are typically measured using an electrical transient response of a testing device compared to a theoretically predicted response using a numerical analysis.

As used herein, the term “binding interaction” or “complex formation” refers to the interaction of a molecule of interest (e.g., a first binding partner or ligand) with another molecule (e.g., a second binding partner or antiligand). Examples of first/second (or ligand/antiligand) binding interactions measurable by the methods and devices of the invention include (1) simple, non-covalent binding, such as dipole-dipole interactions, hydrogen bonding, or Van der Waals interactions, and (2) temporary covalent bond formation, such as often occurs when an enzyme is reacting with its substrate. More specific examples of binding interactions of interest include ligand/receptor (where the ligand may also be a ligand mimic or a mimic analog and a receptor includes an artificial receptor, such as a molecularly imprinted polymer), antigen/antibody, enzyme/substrate, nucleic acids (single and double stranded), DNA/DNA, DNA/RNA, RNA/RNA, nucleic acid mismatches, complementary nucleic acids and nucleic acid/proteins. Binding interactions can occur as primary, secondary, or higher order binding interactions. A primary binding interaction is defined as a first molecule binding (specifically or non-specifically) to a second molecule to form a first molecular interaction complex; a secondary binding interaction is defined as a second molecule binding (specifically or non-specifically) to the first molecular interaction complex; and so on for higher order binding events. The product of first binding partner/second second binding partner (ligand/antiligand) binding interaction is a complex.

The method and device of the invention are particularly useful in detecting binding interactions of biological and pharmaceutical importance that occur in physiological situations (such as in a cellular or subcellular membrane or in the cytosol of a cell). Binding interactions include those that exist under physiological conditions, such as would be present in a natural cellular or intercellular environment, or in an artificial environment, such as in an aqueous buffer, designed to mimic a physiological condition. Local physiological conditions vary within cells and organisms and artificial conditions designed to mimic such conditions can also vary considerably. For example, a binding interaction may occur between a protein and a ligand in a subcellular compartment in the presence of helper proteins and small molecules that affect binding. Such conditions may differ greatly from the physiological conditions in serum, exemplified by the artificial medium referred to as “normal phosphate buffered saline” or PBS. In some embodiment, conditions for performing the measurements in the methods of the invention will be aqueous solutions, although some amounts of organic solvents, such as DMSO, may be present to assist solubility of some components being tested. An “aqueous solution” contains at least 50 wt. % water, preferably at least 80 wt. % water, more preferably at least 90 wt. % water, even more preferably at least 95 wt. % water. Other conditions, such as osmolality, pH, temperature, and pressure, can and will vary considerably in order to mimic local conditions of the intracellular environment in which, for example, a binding event is taking place. The natural conditions in, for example, the cytosol of a cell and a lysosome of that cell, are quite different, and different artificial media would be used to mimic those conditions. Examples of artificial conditions designed to mimic natural ones for the study of various biological events and structures are known. Many artificial media are commercially available.

As used herein, the terms “binding partners,” “ligand/antiligand,” or “complex” and “ligand/antiligand complex” refers to pairs (or larger groups) of molecules that specifically contact (e.g., bind to) each other to form a complex. Such a pair or other grouping typically consists of two or more molecules that are interacting with each other, usually by the formation of non-covalent bonds (such as dipole-dipole interactions, hydrogen bonding, or van der Waals interactions). The time of interaction (sometimes referred to as the on-off time) can vary considerably, even for molecules that have similar binding affinities, as is well known in the art. Examples include antibody-antigen, lectin-carbohydrate, nucleic acid-nucleic acid, and biotin-avidin pairs. Other examples will be readily apparent to those skilled in the art. The term “ligand” is commonly used herein to refer to any molecule for which there exists another molecule (i.e., an “antiligand”) that binds to the ligand, owing to a favorable (i.e., negative) change in free energy upon contact between the ligand and antiligand. There is no limit on the size of the interacting substances; a ligand (or an antiligand) can consist of either an individual molecule or a larger, organized group of molecules, such as would be presented by a cell, cell membrane, organelle, or synthetic analogue thereof. As used herein, “ligand” and “antiligand” both have this broad sense and can be used interchangeably.

Suitable ligands for use in the practice of the invention include antibodies, antigens, nucleic acids (e.g., natural or synthetic DNA, RNA, gDNA, cDNA, mRNA, tRNA, siRNA), lectins, sugars, oligosaccharides, glycoproteins, receptors, growth factors, cytokines, small molecules such as drug candidates (from, for example, a random peptide library, a natural products library, a legacy library, a combinatorial library, an oligosaccharide library and a phage display library), metabolites, drugs of abuse and their metabolic by-products, enzyme substrates including substrate mimics and substrate analogs, enzyme inhibitors, enzyme co-factors such as vitamins, lipids, steroids, metals, oxygen and other gases found in physiologic fluids, cells, cellular constituents, cell membranes and associated structures, cell adhesion molecules, natural products found in plant and animal sources, tumor markers (i.e., molecules associated with tumors), other partially or completely synthetic products, and the like. A “natural ligand” is a ligand which occurs in nature and specifically binds to a particular site(s) on a particular antiligand such as a protein. Ligands also include ligand mimics and ligand analogs. Examples by way of illustration and not limitation include a receptor and a ligand specific for the receptor (e.g., an agonist or antagonist), an enzyme and an inhibitor, substrate or cofactor; and an antibody and an antigen.

An “antiligand” refers to a molecule which specifically or nonspecifically binds another molecule (i.e., a ligand). The antiligand is also detected through its interaction with a ligand to which it specifically binds or by its own characteristic dielectric properties. Alternatively, once an antiligand has bound to a ligand, the resulting antiligand/ligand complex can be considered an antiligand for the purposes of subsequent binding.

As used herein, the terms “complex” and “ligand/antiligand complex” refer to the product of the first binding partner bound to the second binding partner, and the product of the ligand bound to the antiligand, respectively. The binding can be specific or non-specific, and the interacting ligand/antiligand complex are typically bonded to each other through non-covalent forces such as hydrogen bonds, Van der Waals interactions, or other types of molecular interactions.

The term “specifically binds” when referring to a protein or polypeptide, nucleic acid, or receptor or other binding partners described herein, refers to a binding reaction which is determinative of the cognate ligand of interest in a heterogeneous population of proteins and/or other biologics. Under designated conditions (e.g., immunoassay conditions in the case of an antibody), the specified ligand or antibody binds to its particular “target” (e.g., a hormone specifically binds to its receptor) and does not bind in a significant amount to other proteins present in the sample or to other proteins to which the ligand or antibody may come in contact in an organism or in a sample derived from an organism. A ligand that specifically binds to a protein is one that binds at the same site as a natural ligand.

“Polypeptide”, “peptide,” “protein” and “protein target” are used interchangeably to refer to a polymer of amino acid residues. The terms apply to amino acid polymers in which one or more amino acid residue is an artificial chemical analogue of a corresponding naturally occurring amino acid, as well as to naturally occurring amino acid polymers. The protein or protein target to which ligands are being screened in drug discovery methods can be of essentially any type capable of binding some type of ligand including, by way of example and not limitation, enzymes, receptors, antibodies and fragments thereof, hormones, and nucleic acid binding proteins. A protein or peptide may include a particular site, this site is the site at which a ligand and the protein or peptide form a binding complex. For an enzyme, the particular site can be the active site or an allosteric site; in the instance of a receptor, the particular site is the site at which a natural ligand binds.

The term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into an Fab′ monomer. The Fab′ monomer is essentially an Fab with part of the hinge region. While various antibody fragments are defined in terms of the digestion of an intact antibody, one of skill will appreciate that such Fab′ fragments may be synthesized de novo either chemically or by utilizing recombinant DNA methodology. Thus, the term “antibody,” as used herein also includes antibody fragments either produced by the modification of whole antibodies or synthesized de novo using recombinant DNA methodologies. Preferred antibodies include single chain antibodies, more preferably single chain Fv (scFv) antibodies in which a variable heavy and a variable light chain are joined together (directly or through a peptide linker) to form a continuous polypeptide.

A single chain Fv (“scFv” or “scFv”) polypeptide is a covalently linked VH::VL heterodimer which may be expressed from a nucleic acid including VH- and VL-encoding sequences either joined directly or joined by a peptide-encoding linker. A number of structures for converting the naturally aggregated—but chemically separated light and heavy polypeptide chains from an antibody V region into an scFv molecule which will fold into a three dimensional structure substantially similar to the structure of an antigen-binding site.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three-dimensional space to form an antigen binding “surface.” This surface mediates recognition and binding of the target antigen. The three hypervariable regions of each of the heavy and light chains are referred to as “complimentarily determining regions” or “CDRs.”

An “epitope” is that portion of an antigen that interacts with an antibody.

The terms “immunological binding” and “immunological binding properties” refer to the non-covalent interactions of the type which occur between an immunoglobulin molecule (e.g., antibody) and an antigen for which the immunoglobulin is specific.

“Sample” refers to essentially any source from which materials of interest to be analyzed (e.g., ligands and antiligands, such as antibodies and antigens, and nucleic acids and their complements) can be obtained. A sample may be acquired from essentially any organism, including animals and plants, as well as cell cultures, recombinant cells and cell components. Samples can be from a biological tissue, fluid or specimen and may be obtained from a diseased or healthy organism. Samples may include, but are not limited to, sputum, amniotic fluid, blood, blood cells (e.g., white cells), urine, semen, peritoneal fluid, pleural fluid, tissue or fine needle biopsy samples, and tissue homogenates. Samples may also include sections of tissues such as frozen sections taken for histological purposes. Typically, samples are taken from a human. However, samples can be obtained from other mammals also, including by way of example and not limitation, dogs, cats, sheep, cattle, and pigs. The sample may be pretreated as necessary by dilution in an appropriate buffer solution or concentrated, if desired. Any of a number of standard aqueous buffer solutions, employing one of a variety of buffers, such as phosphate, Tris, or the like, preferably at physiological pH can be used.

Biological samples can be derived from patients using well known techniques such as venipuncture, lumbar puncture, fluid sample such as saliva or urine, or tissue biopsy and the like. When the biological material is derived from non-humans, such as commercially relevant livestock, blood and tissue samples are conveniently obtained from livestock processing plants. Similarly, plant material used in the invention may be conveniently derived from agriculture or horticultural sources, and other sources of natural products. Alternatively, a biological sample may be obtained from a cell or blood bank where tissue and/or blood are stored, or from an in vitro source, such as a culture of cells. Techniques for establishing a culture of cells for use as a source for biological materials are well known to those of skill in the art.

As used herein, the term “sample,” “sample mixture,” or “sample solution” refers to the materials being investigated (e.g., the first and second binding partner, ligand and antiligand, and complex and ligand/antiligand complex, if any) and the medium/buffer in which the materials are found. Examples of preferred media are physiologically acceptable buffer solutions.

As used herein, the term “fluid reservoir” refers to where fluid, without regard to physical size or shape, is being maintained in a position that is in electrical communication with the first and second electrode of the cell described herein. “Fluid reservoir” refers to the fluid itself in the cell. In its simplest form, “fluid reservoir” can refer to a fluid droplet or layer formed on a surface and maintained in electrical communication with the first and second electrodes by inertia and/or surface tension. Such arrangements are sometimes used in various “chip” designs commonly used in genomics in which a sample fluid is washed across the surface of a chip. The “fluid reservoir” is contained within a cell having physical walls that restrain movement of the fluid, such as vertical walls that constrain gravitational spreading (as in the side walls of test tube or microtiter plate), completely surrounding walls (as in a sealed container), or partially surrounding walls that direct and/or permit motion in a limited number of directions (such as the walls of a tube or other channel, fluid channels).

The device and method of the invention provide information sufficient to establish whether or not a molecular binding interaction between first and second binding partners (e.g., ligand/antiligand binding) has occurred in a sample by measuring electrically detectable bulk property measurements of a mixture, without requiring separation of the components of the mixture from each other, by establishing a relationship between the bulk property measurements of the mixture and controls. The formation or absence of a complex can be determined based on the nature of this relationship. The signals that are obtained are different from the signal obtained when components are separated measured.

The device and method of the invention are useful for detecting complex formation in a sample resulting from a molecular binding event between first and second binding partners present in the sample. In one embodiment, the device and methods are useful in detecting nucleic acid hybridization (e.g., complex formation between first and second nucleic acids). In another embodiment, the device and methods are useful in detecting immunoreactions (e.g., complex formation between an antibody and an antigen). These embodiments are described below.

Nucleic Acid Hybridization Analysis

In one embodiment, the device and method of the invention are useful for analyzing nucleic acid binding interactions. In these methods, a bulk electrical property sensitive to the dielectric properties of nucleic acids and their complexes, such as hybridization complexes formed between a nucleic acid probe and a nucleic acid target, are measured to detect the absence or presence of hybridized nucleic acids. The methods include diagnostic methods that involve detecting the presence of one or more target nucleic acids in a sample, quantitative methods, kinetic methods, and a variety of other types of analysis such as sequence checking, expression analysis, and de novo sequencing. In the method, nucleic acid binding is detected without the use of labels or separation of sample components.

Certain diagnostic methods that utilize this approach include using a nucleic acid probe that is complementary to a target of known sequence. A sample potentially containing the target of known sequence is contacted with the complementary probe. In some methods, the target and probe are allowed to hybridize and the detection of a response signal is indicative of the sample containing the target of known sequence. Such methods can be used in detecting a single nucleotide polymorphism (SNP). The nucleic acid target containing a polymorphic site includes a first or a second base at the polymorphic site. The nucleic acid probe is selected to be complementary to either a nucleic acid target wherein the polymorphic site includes the first base or is complementary to a nucleic acid target wherein the polymorphic site includes the second base. With knowledge of the sequence of the nucleic acid probe, detection of a response signal makes it possible to identify whether the target contains the first or second base at the polymorphic site.

The method also allows for determining whether a SNP is of the wild type form or a variant form. In this embodiment, the target includes a polymorphic site that can include a first or second base. The nucleic acid probe sequence is selected so that if the target includes the first base at the polymorphic site the target forms a complementary hybridization complex. However, if the target includes the second base at the polymorphic site, then a mismatch hybridization complex is formed. The presence of a complementary signal is indicative of the target including the first base at the polymorphic site, and the presence of a mismatch signal is indicative of the target including the second base at the polymorphic site. Similar approaches can be used when there are more than two allelic forms.

The following definitions are provided for further understanding nucleic acid hybridization analysis by the methods of the invention.

As used herein, “nucleic acid” refers to a deoxyribonucleotide or ribonucleotide polymer in either single- or double-stranded form.

A “polynucleotide” or “oligonucleotide” refers to a single or double-stranded polymer of deoxyribonucleotide or ribonucleotide bases.

A “probe” or “nucleic acid probe” is a nucleic acid capable of binding to a target nucleic acid of complementary sequence through one or more types of chemical bonds, usually through complementary base pairing, usually through hydrogen bond formation, thus forming a duplex structure. A probe can be an oligonucleotide that is a single-stranded DNA. The bases in a probe can be joined by a linkage other than a phosphodiester bond, so long as it does not interfere with hybridization. Probes may be peptide nucleic acids in which the constituent bases are joined by peptide bonds rather than phosphodiester linkages. Some probes may have leading and/or trailing sequences of noncomplementarity flanking a region of complementarity.

A “perfectly matched probe” has a sequence perfectly complementary to a particular target sequence. Such a probe is typically perfectly complementary to a portion (subsequence) of the target sequence.

The term “mismatch probe” refers to probes whose sequence is not perfectly complementary to a particular target sequence.

“Hybridization” refers to binding between a nucleic acid probe and a target sequence via complementary base pairing; the resulting complex is referred to as a “hybridization complex”. A hybridization complex may be either a complementary complex or a mismatch complex.

A “complementary complex” is a hybridization complex in which there are no mismatches between the probe and target sequences that comprise the complex.

A “mismatch complex” is a hybridization complex in which there are one or more mismatches between the probe and target sequences that comprise the complex.

A “polymorphism” refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the “reference form” or the “wild type form” and other allelic forms are designated as “alternative alleles” or “variant alleles.” The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

A “single nucleotide polymorphism” occurs at a polymorphic site occupied by a single nucleotide, which is the site of variation between allelic sequences. The site is usually preceded by and followed by highly conserved sequences of the allele (e.g., sequences that vary in less than [fraction ( 1/100)] or [fraction ( 1/1000)] members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Because the device and method of the invention directly detect complex formation with high sensitivity, nucleic acid hybridization is readily detectable by the device and method of the invention.

Protein Binding Analysis

As noted above, in another embodiment, the invention provides a device and method for analyzing protein binding interactions. In the method, protein/ligand complexes are directly detected based on the measurement of electrically detectable properties of the sample containing the complex. The device and method can be used to detect protein binding events in diagnostic applications such as immunoassays, and analytical applications including identifying ligands and screening ligand libraries.

Because the methods involve direct detection of binding events, it is not necessary to use labeled proteins or ligands, thus simplifying the methods and reducing costs relative to other approaches for monitoring protein/ligand binding events. In addition to diagnostic applications in which a protein or ligand of interest can be qualitatively identified by complex formation, the methods can be adapted for rapidly screening molecules that are of potential therapeutic value.

In one embodiment, the invention provides a label-free, immobilization-free immunoassay. In the method, an electrically detectable bulk property is measured for a control solution that includes a first binding partner (e.g., an antibody or an antigen). The electrically detectable bulk property is then measured for a sample that may or may not include a complex resulting from the specific binding interaction of the first binding partner with the second binding partner. The presence or absence of the complex is determined by comparing the measurements. An exemplary immunoassay is described further in Example 1.

For immunoassays in which the sample of interest is analyzed to determine the presence of an antigen (e.g., ligand), the first binding partner in the control solution is an antibody (e.g., antiligand). Alternatively, for immunoassays in which the sample of interest is analyzed to determine the presence of an antibody, the first binding partner in the control solution is an antigen. In certain embodiments, the first binding partner in the control solution is immobilized on a solid phase, such as a bead or particle.

The concentration of analyte detectable in a typical immunoassay using the methods and devices is about 1 femtomolar.

Devices

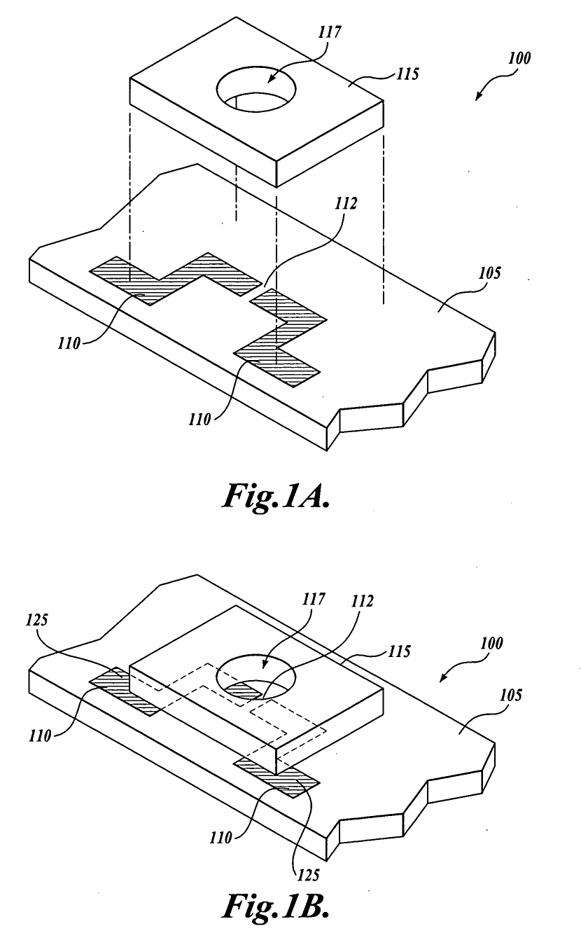

The device of the invention will now be described with reference to FIGS. 1A and 1B. A portion of a device 100 is illustrated (partially exploded) in FIG. 1A and includes a substrate 105 having electrodes 110 patterned thereon. An electrode gap 112 separates the electrodes 110. A structure 115 is disposed over the electrode gap 112 with an aperture defining a well 117.

Suitable substrate materials include organic and inorganic materials and polymers. In a representative embodiment, the substrate 105 is a silicon wafer having an oxidized surface. The electrodes 110 are typically metal, but it will be appreciated that electrically conductive materials of any type, including conductive polymers, are also useful in the device 100. In one embodiment, the electrode gap 112 width is in the range from about 10 nanometers to about 1 millimeter. In another embodiment, the electrode gap 112 width is in the range from about 10 nanometers to about 1 micrometer. In another embodiment, the electrode gap 112 width is in the range from about 10 nanometers to about 500 nanometers. In another embodiment, the electrode gap 112 width is in the range from about 50 nanometers to about 100 nanometers. As the surface area of the electrodes decreases, the sensitivity of the device increases due to the decrease of the electrical double layer effect. Nanoscale electrodes are preferred for high sensitivity applications. The electrodes 110 and the electrode gap 112 are typically created by lithographic techniques known to those of skill in the art, including photolithography, shadow mask lithography, and soft lithography.

The structure 115 is positioned and adapted to retain a fluid therein, the fluid being in contact with the electrodes 110. Exemplary microfluidic devices are described in Example 4. The structure may be adapted to define one or more wells, channels, and other microfluidic elements suitable to the particular application. The structure 115 can be formed from any material, but is typically a polymer, for example, polydimethylsiloxane (PDMS), and the structure 115 and well 117 can be created using lithographic techniques, mechanical techniques, or other methods known to those of skill in the art.

Referring now to FIG. 1B, the assembled device 100 is shown, including the substrate 105 and electrodes 110 upon which the structure 115 is immobilized. The well 117 is positioned such that at least a portion of the electrode gap 112 is exposed within the well 117.

The electrodes 110 include leads 125, which are portions of the electrodes 110 that project further than the structure 115 on the substrate 105, thus allowing for connection between the electrodes 110 and an appropriate device for delivering and analyzing electronic signals (not shown).

In this embodiment, the structure 115 is formed of PDMS, and immobilized on a silicon/silicon dioxide substrate 105, wherein the structure 115 is attached to the surface using a stamp-and-stick method, or other method known to those of skill in the art for immobilizing polymers on a rigid surface.

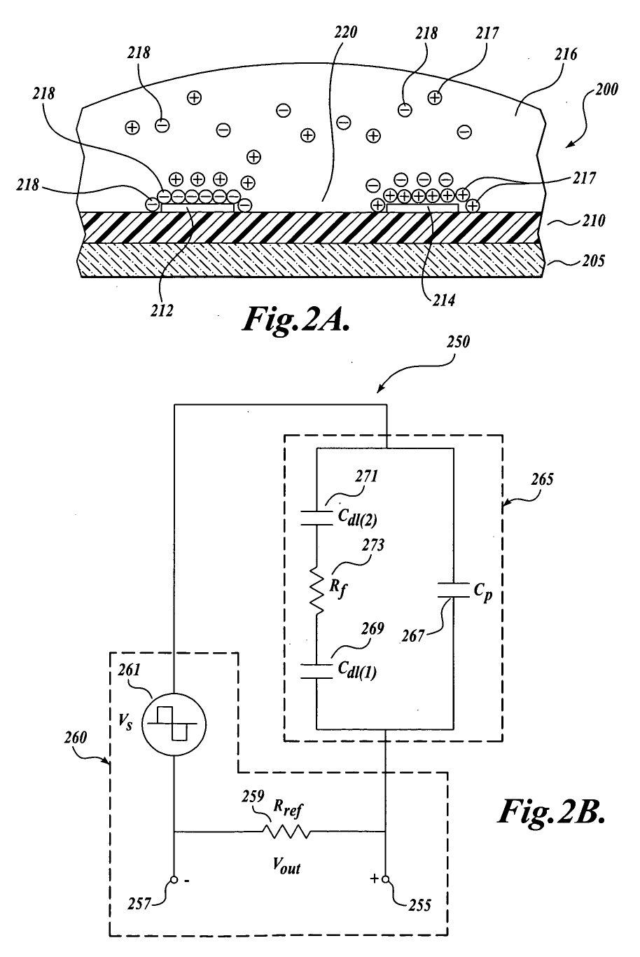

Referring now to FIG. 2A, an idealized cross-section of a device is illustrated so as to better understand the operation of the device 120. In FIG. 2A, the device cross-section 200 includes a silicon substrate 205, a silicon dioxide insulating layer 210, an anode 212 (positive electrode), and cathode 214 (negative electrode) formed from a metal and disposed upon the insulating layer 210. The anode 212 and cathode 214 are separated by an electrode gap 220. In FIG. 2A, cations 217 and anions 218 are represented as positive and negative spheres, respectively, suspended in a fluid 216. No analyte is shown in FIG. 2A, as only the basic device structure is illustrated.

Referring now to FIG. 2B, a circuit diagram 250 illustrates an equivalent electrical circuit used to model the device 200 shown in FIG. 2A, connected to a voltage source. The circuit diagram 250 has a signal processing portion 260 that includes a positive test terminal 255 and negative test terminal 257 connected with a resistor 259 (R_{ref}) and connected to a voltage source 261 (Vₛ). In the representative embodiment illustrated in FIG. 2B, the voltage source 261 is a square wave, low frequency alternating current voltage source.

The circuit diagram 250 also includes a device portion 265 that models the behavior of the device 200 illustrated in FIG. 2A. The device portion 265 includes a parasitic capacitance 267 (Cₚ) representing the capacitance between the anode 212, cathode 214, and insulating layer 210 of the substrate.

Additionally, the device portion 265 includes a model of the behavior of the electrode gap 220 between the anode 212 and cathode 214. The electrode gap 220 includes cations 217, anions 218, and a fluid 216. The device portion 265 includes capacitances 269 and 271 modeling the electrical double layer (EDL) resulting from the accumulation of ions 218 and 217 from the fluid 216 at the anode 212 and cathode 214. In the model circuit diagram 250, the anode 212 capacitance 269 (C_{dl(1)}) and cathode 214 capacitance 271 (C_{dl(2)}) are separated by a resistance 273 (R_{f}) that represents the resistance of the fluid 216 itself.

In operation, the device interrogates the electrode gap 220 by monitoring the electrodes, and uses the transient data to determine the fluid resistance 273 in the electrode gap 220, as discussed below. Knowing the fluid resistance 273 and the geometry of the electrodes, the resistivity of the fluid sample can be calculated. Based on the change in resistivity between samples having differing levels of binding interactions, the fluid resistance 273 will increase or decrease relative to the number of binding events or number of particles present in the electrode gap 220. By calibrating and using the fluid resistance 273 measurement, the invention provides devices and methods capable of binding event detection, typically to the femtomolar level.

The sensitivity of the device can be affected by the surface area of the electrodes in the well. As described further in Example 2, reducing electrode surface area while maximizing electrode gap length results in more sensitive devices due to the attendant reduction in the EDL-related capacitances.

Additionally, the device can be used as a particle counter if the particles affect the resistivity of the fluid based on concentration. An exemplary use of the device as a polystyrene microsphere particle counter is described in Example 3.

An exemplary input signal from the voltage source 261 is a square wave having a frequency of from about 0.01-100 Hz. DC signals and AC signals having non-square shapes are also useful. The output voltage of the device is measured across the positive test terminal 255 and negative test terminal 257. The transient response to the input signal is measured and the resistance can be determined using methods described herein.

Determining the resistance of the fluid is accomplished with the assistance of the modeled circuit described in FIG. 2B and the analysis of the modeled circuit using numerical modeling methods. The reference resistance 259 (R_{ref}) is a known value that may be selected to achieve a desired sensitivity of the device. Thus, three unknown values (Cₚ, C_{dl}, and R_{f}) must be determined to solve the circuit.

The parasitic capacitance (Cₚ) may be determined by measurement with a capacitance meter without solution in a device (e.g., in air).

The final two variables C_{dl}, and R_{f} can be determined through mathematical modeling of the electrical characteristics of the device. The impedance Z_{eq} of the device illustrated in FIG. 2B is characterized in Equation 1 for an input signal Vₛ, where s is the Laplace transformation variable.

$$\frac{1}{{Z}_{eq}} = {C}_{p} s + \frac{1}{{R}_{f} + 2 / {C}_{dl} s} \qquad ( 1 )$$

The corresponding transfer function H(s) for the system is characterized in Equation 2:

$$H ( s ) = \frac{{V}_{out}}{{V}_{s}} = \frac{{R}_{ref}}{{R}_{ref} + {Z}_{eq}} . \qquad ( 1 )$$

A simulated output signal for a device modeled using Equation 2 is used for comparison to the measured transient response of a device to determine resistivity of the fluid in the electrode gap. A representative simulated output signal is determined commercial mathematics software program (such as MATLAB) for a range of R_{f} and C_{dl} values (i.e., a number of curves are generated having different R_{f} and C_{dl} values). The experimental (i.e., actual) transient output signal for the device is measured and a numerical analysis (e.g., the least squares method) is used to determine the modeled output curve that best approximates the experimental output signal. The R_{f} and C_{dl} values of the fitted curve are determined to be those R_{f} and C_{dl} values for the experimental system of the device. From the R_{f} and C_{dl} values, the electrical properties (e.g., resistivity, conductivity, permittivity, and dielectric constant) of the fluid in the electrode gap of the device can be determined using methods known to those of skill in the art (typically in conjunction with the known electrode geometries, including electrode gap width and area).

When the resistance (or other bulk property) of a first sample is known, it can be compared to the resistance of a second sample, with a change in the resistivity between the samples indicating the samples have different compositions. A change in resistivity is useful for detecting binding events and particle concentration, in particular.

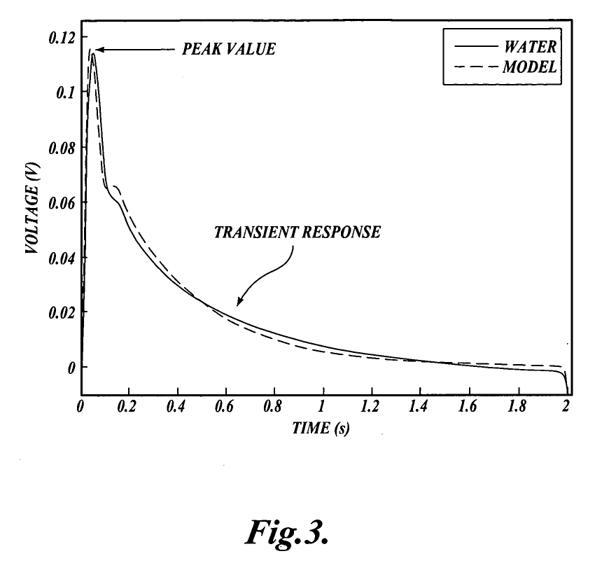

In a representative example, the experimental and theoretical transient responses of a device having water as the sample solution were compared (e.g., for FIG. 2A, the fluid 216 was water in this example). The resulting comparison of theoretical and experimental results is illustrated in FIG. 3, where the experimental data is listed as “water,” and the computer generator data is listed as “MODEL.” The exponential decay shape to the data in FIG. 3 is the result of the R^{C} constant of the device.

Several characteristics of the measured electrical signal of the device can be used to determine the resistance of the fluid, including the peak voltage, root-mean-square (RMS) of the transient response, steady state voltage, and the transition voltage.

The peak voltage is the largest voltage change measured for an AC input signal.

The RMS transient response measurement is determined by the RMS voltage averaged over several voltage cycles. The transient response is less sensitive to small changes in parasitic capacitance (Cₚ) than the peak voltage measurement. The RMS value provides information about the overall variation in the capacitance and resistance, as opposed to the peak value which may overemphasize certain measurements of a system of the device. The RMS voltage output by the device is useful to selectively compare the fluid resistance (R_{f}) and the capacitance of the EDL (C_{dl}), which are typically the properties of interest in a device.

The steady-state voltage is the voltage measured immediately before the voltage alternates. The transition voltage is the voltage at which a characteristic shoulder appears in the transient response, as illustrated in FIG. 3 at about 0.2 seconds.

It will be appreciated that other measurement methods can be used to measure the resistance of the fluid that have not been discussed herein. For example, current or RC constants measurements, which can be used to determine resistance, are known to those of skill in the art.

The following examples are provided for the purpose of illustrating, not limiting, the invention.

### EXAMPLES

### Example 1

### Detecting Antibody/Antigen Reaction

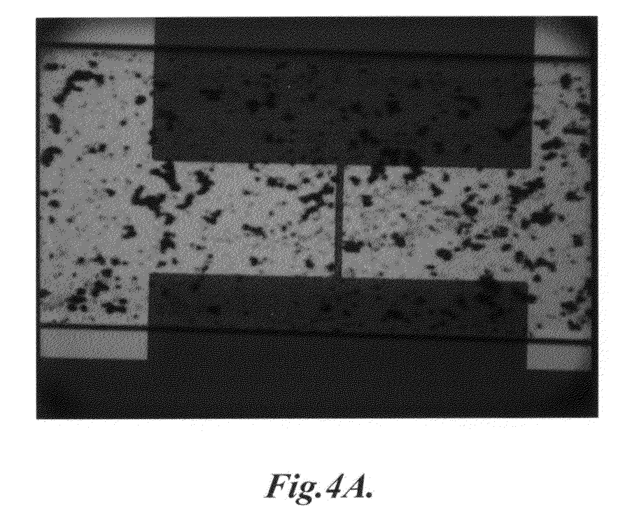

A representative use for the device is the detection of an antibody-antigen (A_{b}A_{g}) reaction. A typical A_{b}A_{g} reaction is performed using a commercial agglutination assay kit (e.g., using E. coli 0157) and a monoclonal antibody against E. coli 0157. Such a test is available in a kit (such as from Pro-Lab Diagnostics, Austin, Tex.). In this representative example, antibody-coated polystyrene beads form complexes that turn black in the presence of an analyte (E. coli). The results of a typical experiment are illustrated in FIG. 4A which shows an optical micrograph of a device having complexed antibody/antigen E. coli agglutination.

In addition to complexing, the antibody-antigen reaction also affects the resistivity of the fluid intermediate the electrodes of the device, and the RMS voltage (using a 2 V, peak-to-peak square wave at 0.25 Hz) can be analyzed to identify the presence of an antibody-antigen binding event. FIG. 4B is a graph illustrating the concentration of antibody-antigen complexes measured in a representative device based on the RMS voltage. The device detects the concentration of binding events and thus acts as a detector for the binding event and quantifies the amount of binding events in the fluid.

### Example 2

### Electrode Surface Area Effects on Device Sensitivity

The position of the electrodes in the device can impact the sensitivity of the device in detecting binding events. In this representative example, the well positioned above the electrode gap of the device is shifted in relation to the electrode gap, and the device performance of different well positions is compared. The analysis results in the conclusion that the device sensitivity increases when the electrode area exposed to the solution is minimized while maintaining a maximum area of electrode gap between the electrodes.

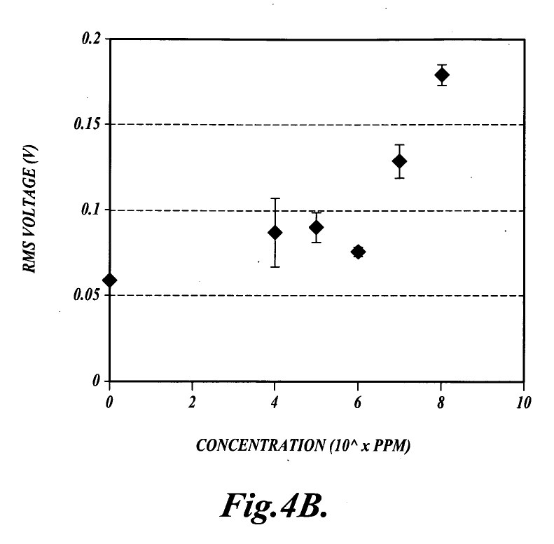

Referring now to FIGS. 5A-5C, micrographs of a representative device are illustrated showing the electrode gap and electrode leads (connecting the electrode gap to diagnostic equipment) in relation to the circular well. Position 1 is illustrated in FIG. 5A, and the electrode gap is justified to the left of the well with the electrode leads extending across the diameter of the well. Position 1 maximizes the amount of electrode surface area exposed in the well.

Position 2 illustrates a device where the electrode surface area is less than that of Position 1, yet a substantial amount of electrode surface area remains exposed in the fluidic well. Position 3 illustrates a minimization of electrode surface area while maintaining a maximum electrode gap area.

Using water as the fluid, the resistivity across the electrodes of the device was tested for each of the three electrode positions in the well (using a 2 V, peak-to-peak square wave at 0.25 Hz). The transient response was analyzed with the model described above in relation to FIG. 3 in order to determine the capacitance and resistance of the device at each position using the RMS method described above.

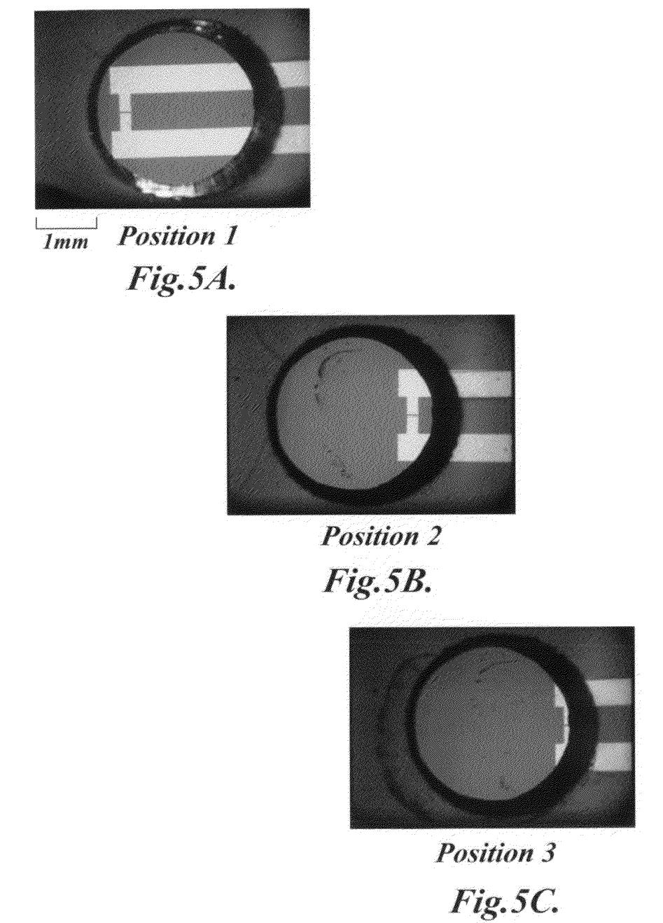

Referring now to FIG. 6, a graph is shown illustrating devices tested using the three well positions and the testing of three parameters (C_{dl}, Cₚ, and R_{f}). As the surface area of the electrodes exposed to the solution in the fluidic well decreased, the EDL capacitance (C_{dl}) decreased, and the fluid resistance (R_{f}) increased. A decrease in the total electrode surface area reduced the total EDL area, and thus the EDL capacitance. The electrode surface area reduction limited the current flow and thus increased the fluid resistance (R_{f}). The parasitic capacitance (Cₚ) also decreased as the total electrode area decreased.

According to the results illustrated in FIG. 6, well Position 3 (illustrated in FIG. 5C) provided the highest sensitivity for the devices tested because the fluid resistance was affected more significantly when the total surface area of electrodes in the device was minimized, thus reducing EDL capacitance.

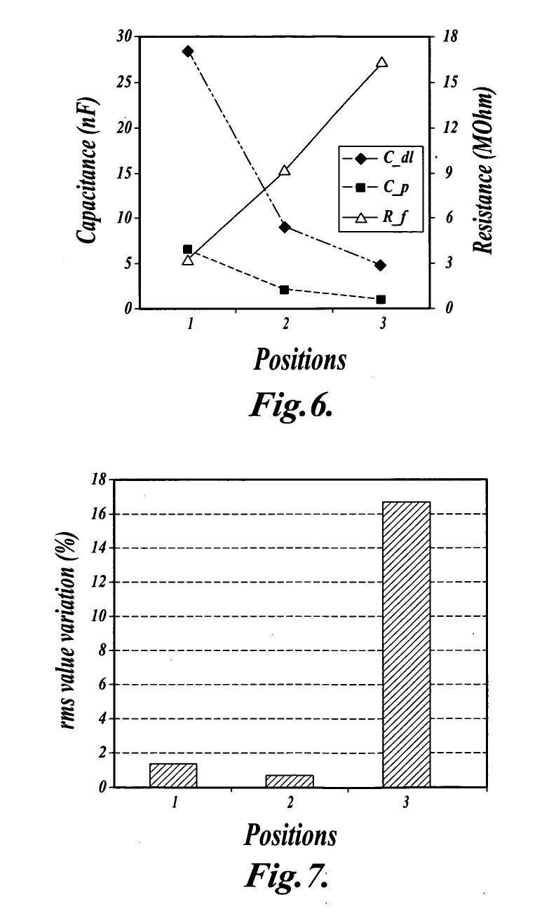

The sensitivity of the three devices illustrated in FIGS. 5A-5C with regard to an antibody-antigen reaction were also tested, and the results are displayed in FIG. 7. In the experiments, the positive antibody-antigen solution included the antigen E. coli 0157:H7 (flagella) and its corresponding antibody coated onto latex beads. A 6 microliter drop of solution was placed in the well, and a square-wave input signal of 2 volts at 0.25 Hz was applied across the electrodes. The output signal was passed through a fourth-order, 10 Hz low-pass Butterworth filter having a 1 MΩ resistance (i.e., R_{ref}). The system was allowed to reach a steady state (typically after approximately 10 seconds) before analysis. FIG. 6 shows the percent difference of the RMS values between a positive (antibody-antigen binding event) and negative control (latex beads having no antibody) of the immunoreaction for three positions. The immunoreaction was clearly detected at Position 3, while the reactions at Positions 1 and 2 were only subtly detected. Thus, Position 3 resulted in the highest sensitivity and had the largest percentage change.

### Example 3

### Particle Counting

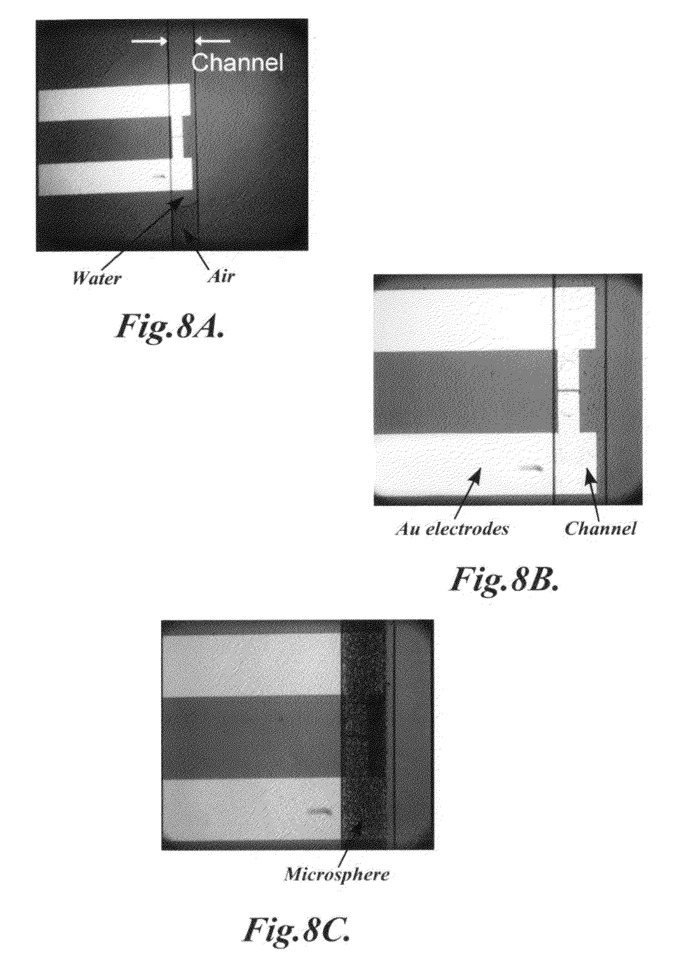

The device can be used for particle counting in addition to measuring binding events. FIG. 8A is a micrograph of a typical device useful as a particle counter, which includes gold electrodes having a 5 micrometer gap separating the electrodes. A microfluidic channel (similar to a well) is patterned passing over the electrodes, and is manufactured from a polymer, such as PDMS. In the micrograph of FIG. 8A, water is present in the channel above the electrodes, and a pocket of air is also shown. In this representative example, the channel is about 500 micrometers in width and 40 micrometers in height.

In the representative example of a particle counting device, 6 micrometer diameter polystyrene microspheres were suspended in water, and a total of three solutions were tested: pure water, diluted microspheres (1.2×10⁵ particles per 10 microliters, comparable to 20 fM), and concentrated microspheres (6×10⁶ particles per 10 microliters, comparable to 1 pM).

The three solutions were controlled by a peristaltic pump and flowed through the channel and across the electrode gap. Micrographs shown in FIGS. 8A-8C illustrate the three solutions used in this representative example. As described above, FIG. 8A illustrates pure water traveling through the channel. FIG. 8B illustrates the diluted microspheres traveling through the channel, and FIG. 8C illustrates the concentrated microspheres traveling through the channel and across the electrode gap.

In this exemplary experiment, the voltage signal was a 1 volt square wave signal driven at 1 Hz and measured across a 1 MΩ resistor. Initially, the water was measured and then removed with the peristaltic pump; next, the diluted microsphere suspension was injected into the same channel followed by a voltage measurement. Similarly, the concentrated microsphere suspension was injected into the channel after the diluted microsphere suspension was removed.

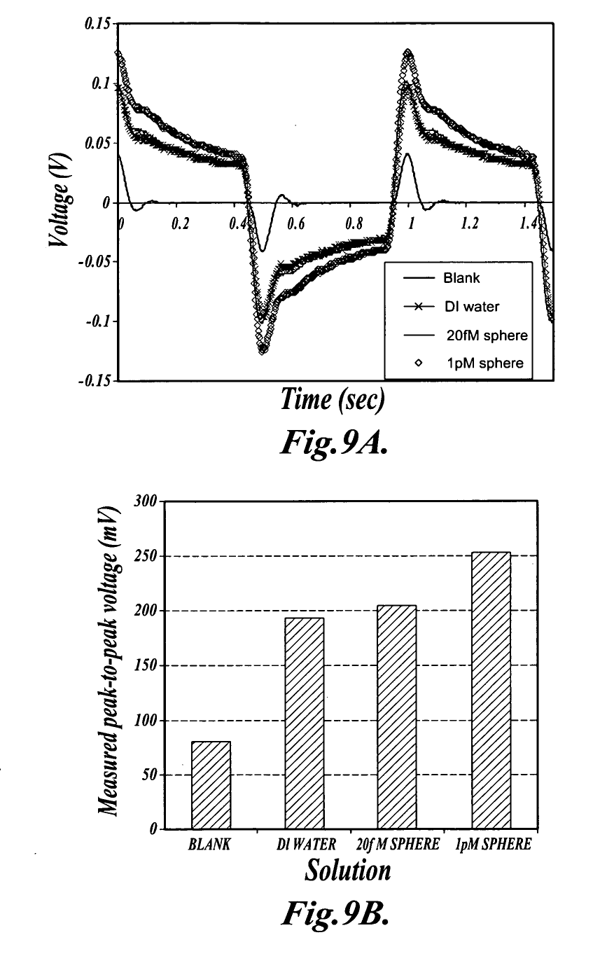

The results of voltage measurements across the electrode gap in the three above-described solutions (water and two microsphere concentrations) are illustrated in FIGS. 9A and 9B. Referring now to FIG. 9A, the peak-to-peak voltage signal measured in air (“Blank”), water (“DI water”), diluted microsphere solution (20 fM), and concentrated microsphere solution (1 pM). In FIG. 9A, as the concentration of the microspheres increases, the peak-to-peak voltage changes, as well as the voltage of the entire transient signals. FIG. 9B graphically illustrates the measured peak-to-peak voltages. The voltage across the electrode gap increase with increasing particle concentration. The increase in peak-to-peak voltage indicates a decrease in the resistance of the fluid between the electrodes, and indicates that the sensitivity of the device is such that even without a binding event, an increase in particle density can be measured using the resistance change in fluid situated in the electrode gap between the electrodes. It is estimated in the representative example described above that the noise level of the system was about 2 mV and thus a minimum measurable concentration of the microsphere suspension would be about 1 fM.

### Example 4

### Microfluidic and Probe Devices

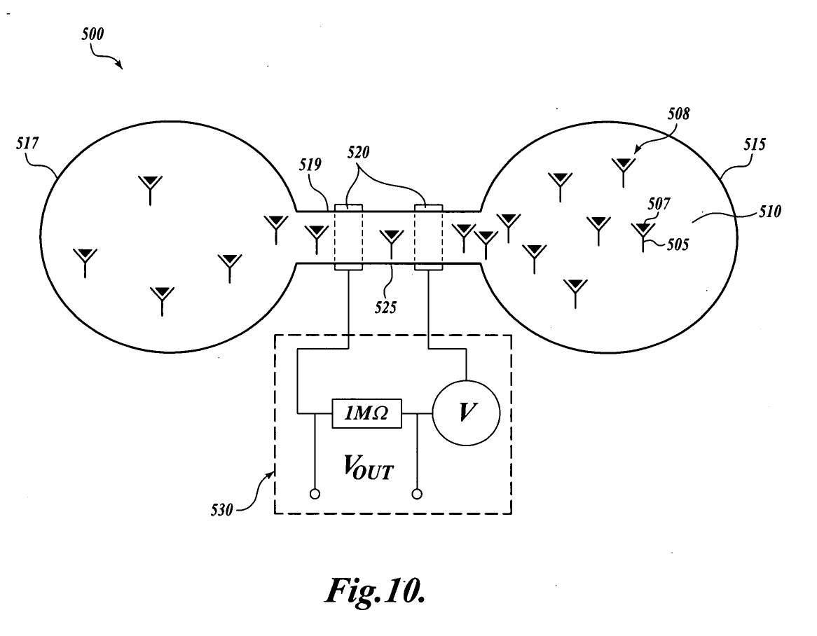

It will be appreciated that devices described herein can be incorporated into larger microfluidic device systems that include components for storing, delivering, mixing, and otherwise processing solutions, as well as delivering such solutions to devices as described herein for testing of resistivity between electrodes. An exemplary microfluidic device is diagrammatically illustrated in FIG. 10.

Referring now to FIG. 10, an exemplary microfluidic device 500 is illustrated having a first chamber 515 and second chamber 517, both containing the fluid 510. In this exemplary embodiment of a microfluidic device, antibodies 505 and antigens 507 form immunocomplexes 508 and are diagrammatically illustrated and are suspended in the fluid 510. A channel 519 connects the first chamber 515 to the second chamber 517 and allows the antibodies 505 and antigens 507 suspended in the fluid 510 to pass over the electrodes 520 and electrode gap 525. As described previously, the electrodes 520 are connected to controlling electronics 530 capable of delivering a voltage signal to the electrodes 520 and measuring the resulting resistance across the electrodes 520 and, thus, the resistance of the fluid 510 disposed in the electrode gap 525.

A number of schemes will be recognized by those of skill in the art as potential uses for the described microfluidic device 500 to test for the presence and/or concentration of complexed antibody 505 and antigen 507 species. In one exemplary method for operating the microfluidic device 500, the fluid 510 initially has only one of either the antibodies 505 or antigens 507 suspended and present in the electrode gap 525. The resistance in the electrode gap 525 would be measured by the controlling electronics 530, after which the complementary immunospecies (i.e., the other of either an antibody 505 or antigen 507) would be introduced into the fluid 510, and if an immunocomplex 508 is formed between the antibodies 505 and antigens 507, the resistivity of the fluid 510 in the electrode gap 525 would change accordingly, and the measured change would indicate not only the presence of immunocomplexes 508 but also their concentration.

It will be appreciated that the microfluidic device 500 illustrated in FIG. 10 is one of an almost unlimited number of configurations of microfluidic devices that can include the device of the invention.

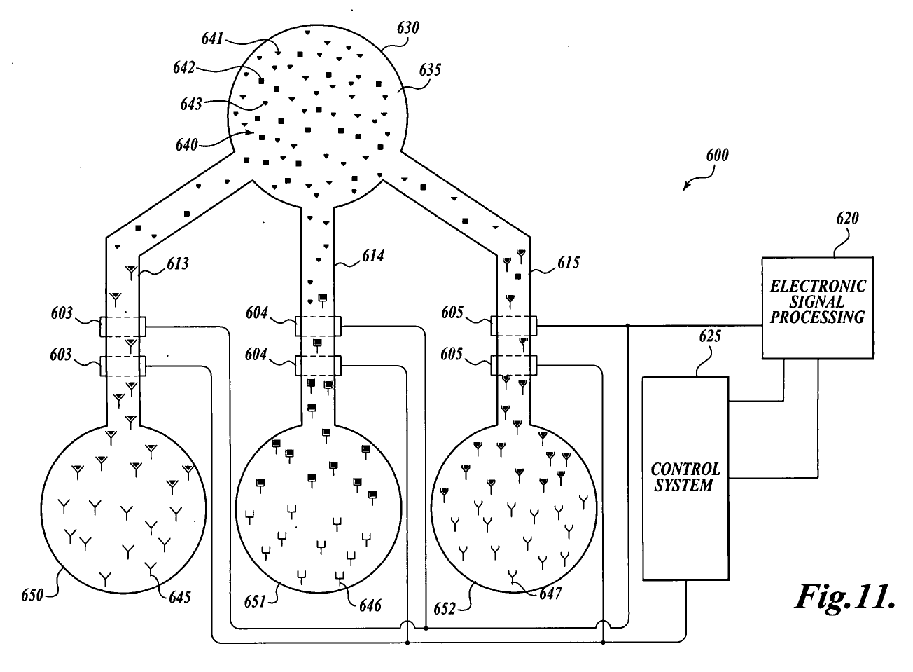

A second exemplary microfluidic device 600 is illustrated in FIG. 11 and includes three electrode sets 603, 604, and 605 disposed in channels 613, 614, and 615 and connected to both an electronic signal processing system 620 and a control system 625 that is also connected to the electronic signal processing system 620. A primary fluidic reservoir 630 contains a fluid 635 that includes a suspension of, in this representative embodiment, antigens 640 including a first antigen 641, a second antigen 642, and a third antigen 643. The primary fluidic reservoir 630 includes an outlet leading to channels 613, 614, and 615, into which the antigens 640 can be directed using microfluidic techniques known to those of skill in the art. Secondary reservoirs 650, 651, and 652 include antibodies 645, 646, and 647 complementary to antigens 641, 642, and 643, respectively. Upon mixing of the fluid 635 containing antigens 640 and the antibodies 645, 646, and 647, immunoreactions may occur, changing the resistance of the fluid 635 at the electrodes 603, 604, and 605.

In the exemplary embodiment of FIG. 11, the antibodies and antigens are illustrated such that antibody 645 will react with antigen 641, antibody 646 will react with antigen 642, and antibody 647 will react with antigen 643. Using the control system 625 and the electronic signal processing 620, the electrodes 603, 604, and 605 at each channel 613, 614, and 615 can be analyzed for resistance so as to detect the immunoreaction that may be present in each channel 613, 614, and 615. For example, in chamber 650, antibody 645 is present, and if antigen 641 is present in the antigens 640, as the fluid 635 flows into channel 613, the mixing of antibody 645 and antigen 641 will create an immunoreaction detectable by electrodes 603 as controlled by the control system 625 and the electronic signal processing 620. While other antigens 642 and 643 may be present in the fluid 635 in channel 613, only the immunoreaction between antibody 645 and antigen 641 will be measured. Thus, each channel 613, 614, and 615 provides analysis and detection for one type of antibody-antigen reaction. It will be appreciated by those of skill in the art that this is one of several possible multiplexed microfluidic device configurations.

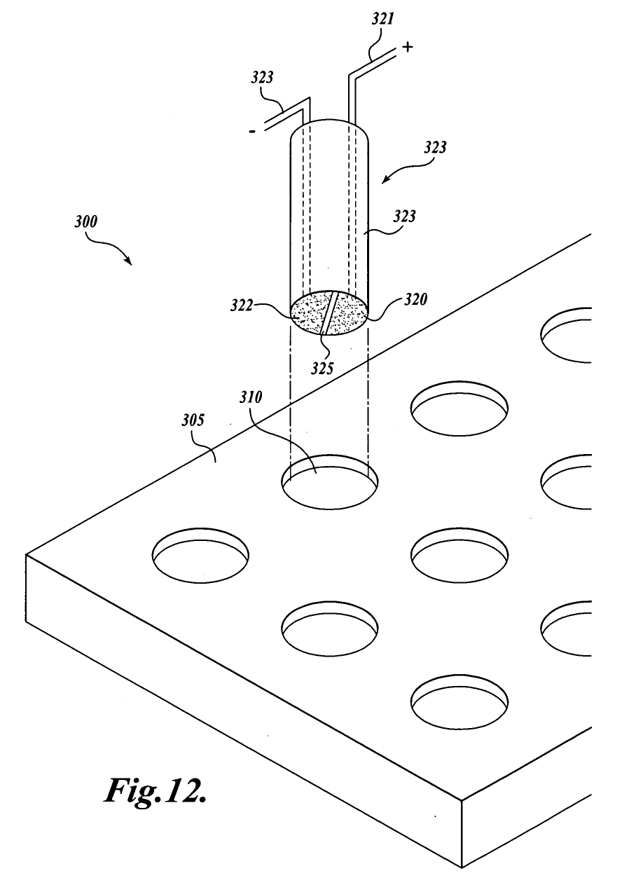

Additionally, a probe-based system can be used instead of confining the electrodes of the device to a fluidic structure (e.g., well or channel). In the representative embodiment illustrated in FIG. 12, a probe and well system 300 shown. The system 300 includes a substrate 305 having wells 310 that may contain a fluidic sample to be measured. A probe 315 includes a probe body 317 supporting a first electrode 320 connected to a positive terminal 321 and a second electrode 322 connected to a negative terminal 323. The first electrode 320 and the second electrode 322 are separated by an electrode gap 325.

As opposed to the other exemplary embodiments described above, the probe-based system 300 has fluidic structures (e.g., well 310) that have no electrodes. The electrode-containing probe 315 is immersed in a fluid in the well 310 and the electrical characterization of the fluid in the electrode gap 325 proceeds as described above.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for detecting a complex, comprising:
(a) providing a testing apparatus defining a well on a substrate, the fluidic structure having a surface with a first electrode, a second electrode, and an electrode gap therebetween;
(b) introducing a sample solution into the well;
(c) applying a voltage across the first and second electrodes;
(d) measuring a transient electrical response to the applied voltage;
(e) estimating a fluid resistance between the electrodes based on the measured transient electrical response;
(f) calculating a resistivity of the sample solution based on the estimated fluid resistance; and
(g) using the calculated resistivity of the sample solution to determine if a complex has been formed in the sample solution.

2. The method of claim 1, wherein estimating the fluid resistance between the electrodes comprises comparing the transient electrical response to transient electrical responses calculated from an analytical model of the testing apparatus.

3. The method of claim 2, wherein the analytical model of the testing apparatus includes a capacitance modeling the electrical double layer capacitance at the electrodes.

4. The method of claim 3, wherein the analytical model of the testing apparatus further includes a capacitance modeling the parasitic capacitance.

5. The method of claim 1, wherein using the calculated resistivity of the sample solution to determine if a complex has been formed in the sample solution comprises comparing the calculated fluid resistivity to a predetermined fluid resistivity correlation.

6. The method of claim 5, wherein the predetermined fluid resistivity correlation comprises a correlation of the resistivity of a control solution that is determined by measuring a transient electrical response of the control solution to an applied voltage and estimating a resistivity of the control solution based on the transient electrical response to the applied voltage.

7. The method of claim 1, wherein the measured transient electrical response is the transient voltage.

8. The method of claim 7, further comprising the step of selecting a parameter of the transient voltage selected from one of the transient voltage RMS value, peak voltage, transition voltage and steady state voltage, and using the selected parameter to determine if a complex has been formed in the sample solution.

9. A method for performing a label-free immunoassay, comprising:
(a) providing a testing apparatus defining a well on a substrate, the fluidic structure having a surface with a first electrode, a second electrode, and an electrode gap therebetween;
(b) introducing a sample solution into the well;
(c) applying a voltage across the first and second electrodes;
(d) measuring a transient electrical response to the applied voltage;
(e) estimating a fluid resistance between the electrodes based on the measured transient electrical response;
(f) calculating a resistivity of the sample solution based on the estimated fluid resistance; and
(g) using the calculated resistivity of the sample solution to determine if antigens are present in the sample solution.

10. A method for detecting a complex, comprising:
(a) combining a first binding partner with a sample to provide a mixture, wherein the mixture comprises a complex formed by a binding interaction between the first binding partner and a second binding partner when the sample comprises the second binding partner;
(b) measuring an electrically detectable bulk property of the mixture; and
(c) determining the presence or absence of the complex in the mixture, and thereby the presence or absence of the second binding partner in the sample, based on the electrically detectable bulk property.

11. The method of claim 10, wherein the electrically detectable bulk property is conductivity or resistivity.

12. The method of claim 10, wherein the electrically detectable bulk property is dielectric constant or permittivity.

13. The method of claim 10, wherein the first binding partner is an antibody or fragment thereof and the second binding partner is an antigen.

14. The method of claim 10, wherein the first binding partner is an antigen and the second binding partner is an antibody or fragment thereof.

15. The method of claim 10, wherein the first binding partner is a first nucleic acid and the second binding partner is a second nucleic acid.

16. The method of claim 10, wherein the first binding partner is an enzyme and the second binding partner is a substrate.

17. The method of claim 10, wherein the first binding partner is a receptor and the second binding partner is a ligand for the receptor.

18. The method of claim 10, wherein the first binding partner is a nucleic acid and the second binding partner is a protein.

19. The method of claim 10, wherein the first binding partner is a cell, cell membrane, or organelle, and the second binding partner is a ligand for the cell, cell membrane, or organelle.

20. The method of claim 10, wherein the first binding partner is immobilized on a solid phase.
